(19) United States
(12) Patent Application Publication
(10) Pub. No.: US 2024/0399009 A1
(43) Pub. Date: Dec. 5, 2024

(54) VOLATILE COMPOSITION DISPENSER WITH REFILLABLE CARTRIDGE AND CLOSE-UP ACTIVATION

(71) Applicant: The Procter & Gamble Company, Cincinnati, OH (US)

(72) Inventors: Rahul VYAS, Singapore (SG); Bhavesh RAJWANI, Singapore (SG); Stefano DEFLORIAN, Trento (IT); Walter SORDO, Trento (IT)

(21) Appl. No.: 18/733,966

(22) Filed: Jun. 5, 2024

Related U.S. Application Data
(60) Provisional application No. 63/470,993, filed on Jun. 5, 2023.
(60) Provisional application No. 63/611,920, filed on Dec. 19, 2023.
(60) Provisional application No. 63/566,971, filed on Mar. 19, 2024.
(60) Provisional application No. 63/567,465, filed on Mar. 20, 2024.

(51) Int. Cl. A61L 9/03

(52) U.S. Cl. CPC A61L 9/03; A61L 2209/133

(57) ABSTRACT

A volatile composition dispenser includes a housing having a first end and an opposing second end, the housing comprising a front frame and a rear frame that are capable of moving away from or toward each other for opening and closing the housing. The rear frame comprises one or more rib elements; and a cartridge configured to be positioned inside the housing. The cartridge includes a reservoir containing at least one liquid volatile composition and a sealing substrate for enclosing the reservoir. The rupture or removal of the sealing substrate enables evaporation of the at least one liquid volatile composition from the cartridge; and the rib elements are configured to apply a compressive force on the cartridge when the housing is in a closed state and the cartridge is positioned within the housing.

### TECHNICAL FIELD

The present disclosure relates to the field of devices and systems for delivering a volatile composition, and particularly relates to a volatile composition dispenser with an openable housing and a replaceable cartridge, as well as related components and methods.

### BACKGROUND

Systems for delivering volatile materials to the atmosphere are well known in the art. Such systems include insect repellents, air fresheners, malodor removal agents, or the like, and function by evaporating a volatile material into a space to deliver a variety of benefits such as air freshening or malodor removal.

Most volatile composition dispensers are configured for one-time use. Typical disposable air freshener dispenser devices are described in PCT Publication No. WO 98/16262 and WO2017/192639, which include an air freshener medium within a container, and a push button actuator which can be manually operated to rupture a foil covering the container for initiating the dispensing of the air freshener into the atmosphere. A problem associated with such a device is that after volatile composition is depleted, it would not be possible to refill or replenish the volatile composition and reactivate the volatile composition dispenser. As a result, the entire product is disposed of, contributing to the environmental problem of plastics waste.

Another problem is that the push button is usually disposed centrally on the cover or shell, and it requires the consumer to forcibly press the button to create enough pressure or cause sufficient deformation of the rupture mechanism so that the peripheral rupture elements (such as pins) are activated to rupture the foil. It may be particularly difficult for younger or elderly consumers to press the push button with sufficient force to activate the device. Further, such push button actuator requires a separate step of activation to be conducted by the consumers before usage, which may not be intuitive to certain consumers (even with clear usage instructions on the package or on the device itself) and may lead to potential consumer confusion as to why the device is not working properly as designed.

Therefore, there exists a need for a volatile composition dispenser with a reusable housing and a replaceable cartridge therein for delivering volatile material. Additionally, it would be beneficial for the volatile composition dispenser to provide an easy-to-use and more intuitive actuation mechanism, as well as improved user satisfaction.

### SUMMARY

The volatile composition dispenser of the present disclosure comprises a reusable housing which is refillable via a cartridge which comprises a volatile composition.

In one example of the present disclosure, the volatile composition dispenser includes a housing having a first end and an opposing second end, the housing including a front frame and a rear frame that are capable of moving away from or toward each other for opening and closing the housing. The rear frame includes one or more rib elements and a cartridge configured to be positioned inside the housing. The cartridge includes a reservoir containing at least one liquid volatile composition and a sealing substrate for enclosing the reservoir. The rupture or removal of the sealing substrate enables evaporation of the at least one liquid volatile composition from the cartridge and the rib elements are configured to apply a compressive force on the cartridge when the housing is in a closed state and the cartridge is positioned within the housing.

In such forms, by means of the openable configuration of the housing and the arrangement of one or more rib elements on the rear frame, the examples of the present disclosure provides advantages that are discussed in detail herein.

### BRIEF DESCRIPTION OF THE DRAWINGS

While the specification concludes with the claims particularly pointing out and distinctly claiming examples of the present disclosure, it is believed that the present disclosure will be better understood from the following description taken in conjunction with the accompanying drawings.

FIG. 1 is a front perspective view of a volatile composition dispenser according to a configuration.

FIG. 2 is a rear perspective view of the volatile composition dispenser shown in FIG. 1.

FIG. 3 is a side perspective exploded view of the volatile composition dispenser shown in FIG. 1.

FIG. 4 is a schematic drawing of a rupture mechanism according to a configuration.

FIG. 5A is a front view of the volatile composition dispenser in an opened state according to a configuration, which housing comprises a through-latch locking structure.

FIG. 5B is a perspective view of the volatile composition dispenser shown in FIG. 5A.

FIG. 6A shows a configuration of a volatile composition dispenser in a locked state, which comprises a button latch.

FIG. 6B is a front view of the volatile composition dispenser shown in FIG. 6A in an open configuration.

FIG. 6C is a perspective view of the volatile composition dispenser shown in FIG. 6B.

FIG. 7 is a side view of a volatile composition dispenser according to a configuration, showing a housing comprising a separation slot.

FIG. 8 is a front view of a volatile composition dispenser according to a configuration, showing a housing comprising separation tabs.

FIG. 9 is a front view of a cartridge according to a configuration, where the cartridge comprises a removable sealing substrate having a pull tab.

FIG. 10 is an illustration of the effect of a compressive force on a fill level of volatile material within a cartridge.

### DETAILED DESCRIPTION

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure is defined solely by the claims. The features illustrated or described in connection with one example configuration may be combined with the features of other example configurations. Such modifications and variations are intended to be included within the scope of the present disclosure.

### Volatile Composition Dispenser

Examples of the present disclosure provide a volatile composition dispenser comprising:
a housing having a first end and an opposing second end, the housing comprising a front frame and a rear frame that are capable of moving away from or toward each other for opening and closing the housing, wherein the rear frame comprises one or more rib elements; and
a cartridge configured to be positioned inside the housing, the cartridge comprising:
   a reservoir containing at least one liquid volatile composition;
   a sealing substrate for enclosing the reservoir,
      wherein:
rupture or removal of the sealing substrate enables evaporation of the at least one liquid volatile composition from the cartridge; and
the rib elements are configured to apply a compressive force on the cartridge when the housing is in a closed state and the cartridge is positioned within the housing.

A volatile composition dispenser can be used to dispense at least one volatile composition and/or other solution or composition, such as a perfume, a fragrance, and/or an insecticide, for example, to an area or atmosphere surrounding the volatile composition dispenser. The volatile composition can comprise a single chemical or a single material that is capable of entering the vapor phase under atmospheric conditions or, more commonly, the volatile composition can comprise a mixture of chemicals and/or materials that are capable of entering the vapor phase under atmospheric conditions.

The volatile composition dispenser may be intended to be used within an interior space, such as an interior space in a building or a passenger compartment of a vehicle, although the present disclosure is not limited to such use. While the volatile composition dispenser will be discussed herein with reference to use within an interior space of a building, such as a room, those of skill in the art will understand that the dispenser can be configured for use in any appropriate environment, and can be configured to dispense any suitable solution, chemical, material, and/or composition.

The volatile composition dispenser may be configured as non-energized volatile composition dispenser for the delivery of a volatile composition in a continuous manner. “Non-energized” can mean that the apparatus is passive and does not require to be powered by a source of external energy. The volatile composition dispenser does not need to be powered by a source of heat, gas, or electrical current, and the volatile composition is generally not delivered by aerosol means.

The continuous emission of the at least one volatile composition can be of any suitable length, such as up to 20 days, 30 days, 40 days, 60 days, 90 days, shorter or longer periods, or any period between 10 to 90 days, for example. Of course, more or less volatile composition can be provided in the volatile composition dispenser to increase or decrease its useful life. Also, the volatile composition dispenser's useful life may be dependent on the conditions (i.e., temperature, pressure, moisture content, airflow etc.) in which it operates.

### Housing

As noted, the volatile composition dispenser comprises a housing having a front frame and an opposing rear frame. The front frame and the rear frame are movable with respect to one another allowing the housing to express a closed position and an open position. The front frame and the rear frame may be pivotally connected to one another or may be completely separable. In some configurations, the front and rear frame may be connected to one another, e.g. pivotally. This advantageously reduces the total number of separate parts, making it easier for a user to replace a cartridge in the volatile composition dispenser. For example, they may hold the housing in one hand and a replacement cartridge in their other hand. In contrast, if the front and rear frame are completely separable then there will be three parts for a user to manage with their two hands.

In order to advantageously improve case of use, the housing may comprise a hinge as described herein (e.g. the housing may comprise a front and rear frame that are pivotally connected). The hinge/pivotal connection enables a user to make use of the lever effect when closing the housing and applying the compressive force described herein.

In some configurations, the connection between the front and rear frames (e.g. a pivot) may provide a degree of resistance for opening and closing the housing (e.g. by friction), such that the front and rear frames are able to hold an open position without external support. This may advantageously improve the case of replacing a cartridge within the housing.

Regardless of the connection between the front frame and the rear frame, the front frame and/or rear frame may comprise separation tabs and/or separation slots which can facilitate grasping by a user of the front frame and/or rear frame and also facilitate separation of the front frame from the rear frame. Where separation tabs are provided on both the front frame and the rear frame, the separation tabs may be spaced from one another to facilitate grasping by a user. For example, one or more separation slots may be included at the interface between the front frame and the rear frame or may be comprised wholly by the front frame or rear frame. Where more than one separation slot is utilized, a first separation slot may be positioned on one side of the housing and a second separation slot may be positioned on an opposite side of the housing.

Where separation tabs are utilized, the front frame may comprise one or more front separation tabs that are spaced from one another, and the rear frame may similarly comprise one or more rear separation tabs that are spaced from one another. In such configurations, the one or more front separation tabs may similarly be spaced from the one or more rear separation tabs.

The housing of the volatile composition dispenser may typically be formed from a plastics material, such as a polypropylene. A person skilled in the art will be aware of other suitable plastics that may be used.

The front frame and rear frame may together comprise a locking structure to releasably interlock the front and rear frames together upon closing of the housing. Thus, the locking structure may releasably lock the housing in a closed state. The locking structure may be disposed at a movable end (e.g. the second end) of the housing, which movable/second end is opposing to a first end in which the front and rear frames may be connected, e.g. via a hinge. Various locking structures can be utilized, including, for example, latches, snap fits, clasps and catches. Representative latch locking structures include through-latch and button latch structures.

The locking structure may be configured to produce a sound of at least 50 dB (such as from 50 dB to 65 dB) upon closing the housing and engaging the locking structure. This sound confirms to a user that the housing has been properly closed and is locked in the closed state. This sound is particularly advantageous when the housing is used with a cartridge that is activated by closing the housing, as is described herein, because the sound confirms to a user that the cartridge has been activated. This overcomes a problem with prior art volatile composition dispensers involving button activation mechanisms where a user was often unsure if a press of the button had properly activated a cartridge.

As mentioned herein, the rear frame may comprise one or more rib elements. The rib elements are configured to apply a compressive force on the cartridge, for example by impacting the cartridge when the housing is in a closed state. Typically, the rib elements are configured to apply the compressive force on a membrane of a cartridge when the housing is in a closed state (whether through direct impact or through one or more additional components or layers).

The rib elements may take the form of one or more protrusions extending from the rear frame towards the middle of the housing. The rib elements may have any appropriate size and shape, such as a cubic, cuboidal, cylindrical, conical or polygonal shape that may have straight or curved edges and faces. The rib elements may have any appropriate size, but may typically have a maximum dimension extending from the rear frame of about 0.1 to 1 cm, and a maximum dimension parallel to the plane of the rear frame of about 0.1 to 2 cm. However, a person skilled in the art will appreciate that larger or smaller rib elements may be used.

In some configurations in which the rib elements are configured to apply a compressive force directly on a membrane during use, the rib elements should be of an appropriate size and shape that does not rupture, pierce, or otherwise damage the membrane.

The front frame may comprise a window portion. The window portion advantageously allows for the cartridge to be visible from outside the volatile composition dispenser, and this may allow for a fill level of volatile composition to be easily determined without opening the volatile composition dispenser. The window portion may be a cut-out portion of the housing (e.g. of the front frame), or the window portion may be a part of the housing (e.g. of the front frame) that is made from a transparent material. In some configurations, the window portion may be configured to receive a part of the cartridge. This helps to securely hold the cartridge in place when the housing is closed, without requiring any adhesive or other adhering means. In some such configurations, the window portion may be a cut-out portion of the front frame, which is configured to receive a part of the cartridge (e.g. the reservoir).

The window portion may have any appropriate shape. In order to hold the cartridge securely and prevent rotation of the cartridge, the window may have an oval shape. The window may have any appropriate size. For example, the window may have a maximum dimension of from about 4.5 cm to about 5.5 cm. The window may have an oval shape and an aspect ratio of from about 1.2 to about 1.8, such as about 1.3 to about 1.6.

Therefore, in some configurations, the cartridge may be configured to fit inside a window portion of the front frame. For example, the cartridge may comprise a reservoir portion that substantially conforms to a shape of a window portion of the front frame. The reservoir portion may be configured to extend into a window portion of the front frame by at least 1 mm, at least 2 mm, at least 3 mm, at least 4 mm, or at least 5 mm. In such cases, the reservoir portion may extend all the way through the window portion, or may rest within the window portion. Furthermore, the reservoir portion may substantially conform to a circumferential shape of the window portion. The reservoir portion may be configured to rest within the window portion and have a maximum distance from the perimeter of the window portion of less than 3 mm, such as less than 2 mm, such as less than 1 mm, such as less than 0.5 mm.

In some configurations, the cartridge may be held within the housing such that the cartridge cannot rotate within the housing. This may be the case, for example, where the cartridge is received by a window portion in the housing, which window portion is not circular.

In yet further configurations, the cartridge, when received by the window portion, may be substantially unable to move within a plane of the window portion. For example, the cartridge may have a maximum movement distance within a plane of the window portion of less than 4 mm, such as less than 3 mm, less than 2 mm, or less than 1 mm. In this context, maximum movement distance refers to the longest distance the cartridge is able to travel in a straight line within the plane of the window. This property may be measured while the housing is open, e.g. when the rib elements are not in contact with the cartridge.

To facilitate emission of the volatile composition, the rear frame may comprise one or more apertures which allow evaporated volatile composition to exit the housing. The apertures may be configured as described in U.S. Pat. No. 11,207,440. Additionally, the front frame may similarly comprise one or more apertures which facilitate airflow through the housing. In some configurations, the aperture comprised by the front frame may have a lower aperture area than that of the one or more apertures in the rear frame.

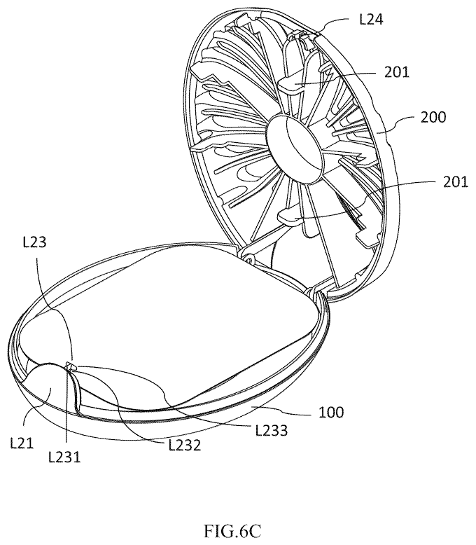

The volatile composition dispenser described herein may advantageously allow a spent or finished cartridge to be removed from the housing without a user needing to touch the cartridge, thereby avoiding contact between a user's hands and organic components of the volatile composition throughout the entire cartridge life cycle. This may be achieved by, for example, opening the housing (as shown in FIGS. 6B and 6C) and upending the volatile composition dispenser so that the cartridge falls out of the housing. This benefit of the examples of the present disclosure is particularly advantageous because a spent or finished cartridge will still contain a small amount of residual volatile composition, and so a membrane will be wetted with organic components even when the cartridge has reached the end of its effective life.

In some configurations described herein, the cartridge is configured to be removed from the housing without being touched by a user. In this context, “without being touched by a user” is to be understood as enabling the removal of the cartridge without a user touching the cartridge, whether directly touching the cartridge, or indirectly touching the cartridge such as with the use of gloves or another implement such as a stick.

Thus, in some configurations, the housing and cartridge may be configured such that when a cartridge located within the housing has reached end of life, the cartridge may be removed from the housing by the steps:
(i) opening the housing; and
(ii) upending the housing,
without a step of disengaging or disadhering the cartridge from the housing.

Therefore, in some configurations, the housing is configured such that when the housing is in an open configuration, a cartridge placed within the housing may be removed by upending the housing.

For example, the cartridge may be configured to be held within the housing without adhesion, and/or without attachment. In this context, “adhesion” refers to the use of an adhesive to secure the cartridge within the housing, for example an adhesive strip such as glue, adhesive tape or Velcro. Thus, in some configurations the cartridge may not comprise an adhesive on an external portion of the cartridge, which adhesive is intended to releasably bond the cartridge to a housing. “Attachment” refers to any mechanism of securing the cartridge within the housing that requires an active releasing or disengaging step and does not allow the cartridge to simply fall out of the housing when the housing is opened and upended. For example, “attachment” might refer to a hook or clamp, or any other attachment means for securing the cartridge to the housing and that would require a disengaging or releasing step. Alternatively, the cartridge may be configured to be held within the housing by virtue of an attachment means that can be released or disengaged without touching the cartridge, such as via a button or slider.

### Cartridge

The volatile composition dispenser comprises a cartridge configured to be positioned inside the housing. The cartridge is typically a single-use disposable cartridge that contains a volatile composition for release to a surrounding environment, and once a cartridge is depleted of volatile composition it may be removed from the housing and replaced by a new cartridge. The depleted cartridge may be disposed of. This configuration (reusable housing with a single-use replaceable cartridge) reduces the amount of material contributed to landfill as compared to an entirely disposable single-use volatile composition dispenser, and also uses a lower volume of material (e.g. plastics) during the manufacturing process. Nevertheless, a skilled person will appreciate that the volatile composition dispenser of the present disclosure may instead comprise a reusable cartridge that is itself refillable, such that a depleted cartridge may be removed from the housing, filled with volatile composition, and then replaced in the housing and reused.

The cartridge may comprise a reservoir, a membrane, and a sealing substrate. In such cases, the reservoir contains the volatile composition and is enclosed by both of the membrane and the sealing substrate, which may enclose the reservoir in any order. Thus, the membrane may enclose the sealing substrate, or the sealing substrate may enclose the membrane.

The cartridge may comprise a seal area as described herein. Thus, the cartridge may comprise an outer peripheral seal area, in which a peripheral portion of the reservoir and membrane may be sealed together. In configurations where the sealing substrate encloses the membrane, a peripheral portion of the sealing substrate may be sealed to a peripheral portion of the membrane at the outer peripheral seal area, i.e. a peripheral portion of each of the reservoir, membrane and scaling substrate may be sealed together at the outer peripheral seal area, provided that the sealing substrate may nevertheless be removed from the cartridge. This may be the case where the edges of the reservoir and membrane are coterminous. Alternatively, the edge of the reservoir may extend beyond the edge of the membrane, and the sealing substrate may be sealed directly to the reservoir at a peripheral region of the reservoir that is beyond the edge of the membrane. In configurations in which the membrane encloses the sealing substrate and the cartridge also comprises a rupture mechanism for rupturing the sealing substrate, the outer peripheral seal area may comprise only a peripheral portion of the reservoir and membrane. In such configurations, the cartridge may also comprise an inner peripheral seal area in which an inner peripheral portion of the reservoir is sealed to the sealing substrate. This may be achieved by providing the reservoir with an intermediate step at an inner peripheral portion (e.g. between the outer peripheral portion and a main body of the reservoir), where the sealing substrate may be sealed to the intermediate step of the reservoir.

The reservoir, membrane, and sealing substrate are discussed in turn below.

### Reservoir

The reservoir contains the volatile composition, and has an opening that is enclosed by the membrane and sealing substrate.

The reservoir of the cartridge may typically be formed from a plastics material, which may advantageously be transparent to allow an easy view of a fill level of volatile composition within the reservoir. An example of a suitable material is polyethylene terephthalate (PET).

In some configurations, the reservoir may be configured to be received by a window portion of the housing, so that the cartridge is held securely within the housing when the housing is closed. In such configurations, it may be advantageous for the reservoir to be formed from a transparent material, so that the fill level of volatile composition within the reservoir is visible from outside the volatile composition dispenser, through the window portion.

The reservoir may have any appropriate shape. In order to hold the cartridge securely and prevent rotation of the cartridge, the reservoir may correspond to a shape of a window in a housing with which the cartridge is configured to be used. Thus, the reservoir may have an oval shape. The combination of a window and reservoir both having an oval shape ensures that the cartridge is held securely within the window and cannot rotate within the window. In addition, the absence of corners/vertices allow for the cartridge to be placed and removed from the window more easily than a polygonal shape which requires specific alignment. When an oval shaped cartridge is placed into an oval shaped window, the curved edges of the cartridge and window will naturally align the cartridge during insertion, improving case of use and user experience. The reservoir may have any appropriate size. For example, the reservoir may have a maximum dimension of from about 4.5 cm to about 5.5 cm. The reservoir may have an oval shape and an aspect ratio of from about 1.2 to about 1.8, such as about 1.3 to about 1.6. These dimensions may be measured at a maximum distance away from the membrane, in a plane that is parallel to the plane of the membrane. The reservoir may comprise a trapezoid-like taper as the reservoir extends away from the membrane (e.g. the reservoir may have a tapering cross-section with increasing distance from the membrane). This taper improves case of insertion into a window of a housing since the taper guides the cartridge into the window during insertion. The reservoir may have a variable depth, which may also increase case of insertion into a window of a housing as a less deep end of the reservoir may be inserted first, and a sloped section of the reservoir may help to align the reservoir within the window during insertion. Thus, the reservoir may have a depth at a first end that is greater than a depth at a second end. In some configurations, the reservoir may comprise a first end section, a second end section at an end opposing the first end section, and a sloped section therebetween. In some configurations, the sloped section may have a slope angle of from 2° to 35°, such as from 3° to 25°, such as from 4° to 15°. The first and second end sections may correspond to the respective end of the cartridge, such that the sloped section extends to and/or from the first and/or second end. Alternatively, the first and/or second end sections may have substantially uniform depth, with the sloped section therebetween. In this context a substantially uniform depth may correspond to a maximum to minimum depth ratio of from about 1 to about 1.4, preferably from about 1 to about 1.3, more preferably from about 1 to about 1.2, where the maximum and minimum depth are measured along a line bisecting the cartridge in a longitudinal direction.

It is worth noting that the depth of the reservoir can be useful, particularly where the scaling substrate is removable—removable sealing substrates are discussed in additional detail hereafter. For example, where the depth of the reservoir is sufficient, a user may grasp a sidewall of the reservoir while removing the sealing substrate. It is believed that a reservoir depth of at least 5 mm, more preferably at least 6 mm, even more preferably at least 7 mm, or most preferably at least 8 mm of depth can provide an adequate gripping surface when a user removes the sealing substrate. While the foregoing represents minimum depths of the reservoir which provide an adequate gripping surface, it is further believed that too high of a value for the reservoir depth can make handling the cartridge challenging. For example, it is believed that the reservoir depth can be 15 mm or less, preferably about 14 mm or less, more preferably about 13 mm or less or most preferably 12 mm or less. Of course, the minimum and maximum depths for the reservoir may be combined to create ranges of depth for the reservoir.

Additionally, as noted previously, where the depth of the reservoir varies along its length and/or width, it may be preferable to have the depth of the reservoir as described above adjacent a pull tab for the sealing substrate. This may improve the case of gripping the cartridge whilst removing the sealing substrate. For example, if the pull-tab is disposed on a side of the cartridge, then a maximum reservoir sidewall depth may be adjacent to the pull-tab, e.g. on the side of the cartridge.

As noted, the reservoir comprises a sidewall. A periphery may extend outboard of the sidewall. The periphery of the cartridge may comprise a sealing area where the membrane and sealing substrate are joined to the reservoir. Where the sealing substrate is removable, an adhesive dead area may be provided in the periphery. In order to provide sufficient adhesion of the membrane and sealing substrate, it is believed that the periphery should have a width (generally perpendicular to the sidewall) of at least 3 mm, more preferably at least 5 mm. In order to facilitate removal of the sealing substrate it is believed that the dead area may have a width of at least 1 mm or more preferably 2 mm, even more preferably at least 2.5 mm, from an outer edge of the periphery toward the sidewall. Greater than 2.5 mm of adhesive dead area is believed to detrimentally affect adhesion of the sealing substrate and membrane which may lead to leakage during shipping, shelving and/or during use.

### Membrane

The volatile composition is in liquid form and is configured to evaporate through a membrane. Accordingly, the cartridge comprises a microporous membrane, which for the sake of brevity may be referred to herein as “the membrane”. The membrane may enclose the reservoir such that volatile composition is unable to escape from the cartridge without passing through the membrane. The membrane may prevent the passage of liquid, such that the volatile composition is only able to escape the cartridge by evaporating through, or from, the membrane.

The membrane is vapor permeable and capable of wicking liquid, yet prevents free flow of liquid out of the membrane. Any suitable membrane may be used. Purely by way of example, certain properties that may result in advantageous membranes are discussed below. However, the present disclosure is not limited to membranes having the properties below, and any membrane known in the art that allows the volatile composition to evaporate may be used in the examples of the present disclosure.

The membrane may have any appropriate volume average pore diameter, such as from 0.01 μm to 0.5 μm, such as from 0.02 μm to 0.3 μm, such as from 0.05 μm to 0.2 μm, more particularly from 0.065 μm to 0.15 μm since this may provide improvements with regard to evaporation rate and controlling leakage or sweating of volatile composition. In certain configurations, the membrane may have a volume average pore diameter of from 0.065 μm to 0.15 μm, from 0.07 to 0.12 μm, from 0.07 to 0.11 μm, or 0.08 to 0.1 μm.

In some configurations, the membrane may have a pore size distribution such that at least 50%, such as at least 60%, such as at least 70%, such as at least 80% or such as at least 90% of the pores of the membrane have a pore diameter of from 0.065 μm to 0.15 μm.

The membrane may comprise (e.g. be formed from) polyethylene, such as ultra-high molecular weight polyethylene (UHMWPE), though other length polyethylene chains may also be used. As used herein, UHMWPE refers to polyethylene having a molecular mass of from about 3.5 million to 7.5 million amu.

The membrane may have a thickness in the z-direction, of about 0.01 mm to about 1 mm, alternatively between about 0.2 mm to about 0.4 mm, from about 0.22 to about 0.37 mm, e.g. from about 0.25 to about 0.35 mm.

The membrane may be formed from a single piece, or single sheet, of material. In other words, the membrane may not be laminated. Thus, the membrane may be formed from a single sheet of polyethylene having a thickness as described above.

Those of ordinary skill in the art will appreciate that the surface area of the membrane can vary depending on the user preferred size of the cartridge. In some configurations, the (evaporative) surface area of the membrane may be about 2 cm² to about 100 cm², alternatively about 10 cm² to about 50 cm², alternatively about 10 cm² to about 45 cm², alternatively about 10 cm² to about 35 cm², alternatively about 15 cm² to about 40 cm², alternatively about 15 cm² to about 35 cm², alternatively about 20 cm² to about 35 cm², alternatively about 30 cm² to about 35 cm², alternatively about 35 cm².

The membrane may have any appropriate porosity. For example, the membrane may have a porosity of from 45% to 70%, on a volume basis, such as from 45% to 65%. In certain configurations, the porosity may be from 50 to 70%, such as 55 to 65%.

The membrane may have any appropriate total pore volume, such as from 0.6 to 2 cm³/g. Typically, the total pore volume may be from 0.65 to 1.6 cm³/g, such as 0.7 to 1.5 cm³/g. In certain configurations, the total pore volume may be from 0.8 to 1.4 cm³/g.

The membrane may have any appropriate bulk density, such as from 0.3 to 0.8 g/cm³. Typically, the bulk density may be from 0.35 to 0.75 g/cm³, such as from 0.4 to 0.7 g/cm³. In certain configurations, the bulk density may be from 0.4 to 0.6 g/cm³.

Suitable membranes for the present disclosure include polyethylene membranes having the properties described herein, available from Microporous, LLC.

The membrane may comprise any suitable filler and plasticizer known in the art. Fillers may include finely divided silica, clays, zeolites, carbonates, charcoals, and mixtures thereof. In one configuration, the membrane may be filled with about 30% to about 80%, by total weight, of silica.

In one aspect of the present disclosure, the membrane may include a dye that is sensitive to the amount of volatile composition it is in contact with to indicate end-of-life. Alternatively, the membrane may change to transparent when in contact with a fragrance or volatile composition to indicate diffusion is occurring. Other means for indicating end-of-life that are known in the art are contemplated for the present disclosure.

The membranes described herein may advantageously provide a clear visual change when wetted with volatile composition, and when dry (whether before use or at end of life). Such visual changes may be more detectible when the membrane does not comprise a white pigment (e.g. TiO₂). Therefore, the membrane may comprise less than 5 wt. % of a white pigment, such as less than 1 wt. % of a white pigment, less than 0.1 wt. % of a white pigment, or less than 0.01 wt. % of a white pigment. The membrane may be free from a white pigment.

The visual change when the membrane is wetted as compared to dry may be more noticeable when the membrane comprises a coloured dye/pigment or a black dye/pigment. Therefore, the membrane may comprise a coloured or black dye/pigment, such as activated charcoal. Such a coloured or black pigment/dye (e.g. activated charcoal) may be present in any suitable amount, such as from 0.1 to 5 wt. %, e.g. 0.3 to 1 wt. %.

### Sealing Substrate

The cartridge comprises a sealing substrate that encloses the reservoir, and hence, encloses the volatile composition. This prevents evaporation of the volatile composition for as long as the scaling substrate is in place and intact. The sealing substrate may be removed or ruptured to allow the volatile composition to evaporate. This removal or rupturing may be referred to herein as “activating” or “activation of” the cartridge. The cartridge is configured to be activated before use, i.e. the sealing substrate is configured to be ruptured or removed before use. In this context, “use” refers to enabling the volatile composition to evaporate from the cartridge, so that the volatile composition dispenser may perform its function of dispensing volatile composition by evaporation.

Non-limiting examples of suitable sealing substrates include an impermeable film, foil, or laminate, such as a flexible (e.g. polymeric) film, a flexible (e.g. metal) foil, or a composite material (e.g. a foil/polymeric film laminate). The impermeable film, foil or laminate is provided adhered to the cartridge to prevent evaporation of volatile composition. A particular example of a suitable scaling substrate is aluminium foil.

The sealing substrate may have any appropriate thickness, such as from 10 μm to 1 mm, from 15 μm to 100 μm, from 18 μm to 50 μm, or from 20 μm to 35 μm.

As mentioned above, there are two possible configurations for the order of the sealing substrate and the membrane.

In some configurations, the sealing substrate may be positioned between the membrane and the volatile composition, preventing the volatile composition from contacting the membrane. In such configurations, the rib elements of the housing may be configured to, upon closure of the housing, cause rupture of the sealing substrate and allow volatile composition to pass through the ruptured sealing substrate and come into contact with the membrane. The volatile composition may then impregnate the membrane, from which it may evaporate. The cartridge may comprise a rupture mechanism, and rupture of the sealing substrate may be caused by actuation of the rupture mechanism by the rib elements. Suitable rupture mechanisms are described in detail in U.S. Pat. Nos. 10,561,754, 10,561,755 and 10,561,756. Alternatively, the rib elements may cause rupture of the sealing substrate directly, by applying pressure to the sealing substrate through the membrane. Thus, the membrane may be capable of deformation such that as the rib elements impact the membrane, the membrane is able to deform in the direction of the applied force (e.g. the compressive force described herein), towards the sealing substrate. In such configurations, the sealing substrate may be formed from a material that is substantially inelastic, such as a metal foil. When a force (e.g. the compressive force described herein) is applied to the membrane, the membrane deforms and comes into contact with the sealing substrate. The sealing substrate is unable to sufficiently deform or flex in the direction of the force, and so may be ruptured by the force. In such configurations, it is important that as the membrane is deformed by the applied force and presses against the scaling substrate, the sealing substrate ruptures before the membrane is damaged. In other words, the force applied by the rib elements causes the sealing substrate to be ruptured, whilst the membrane is deformed without breaking, rupturing or tearing. Thus, in some configurations, the membrane may enclose the sealing substrate, and the sealing substrate may be configured to be ruptured by a force applied to the sealing substrate through the membrane (e.g. by the compressive force applied by the rib elements).

In other configurations, the membrane may be positioned between the volatile composition and the sealing substrate, such that the volatile composition is in contact with the membrane but cannot evaporate and leave the cartridge because the membrane is enclosed by the scaling substrate. In such configurations, the housing, much like the foregoing, may be provided with ribs which directly rupture the scaling substrate; however, this may result in only small holes in the scaling substrate, limiting the rate at which the volatile composition may evaporate from the cartridge, detrimentally affecting the efficacy of the volatile composition dispenser. Alternatively, in such configurations, the sealing substrate may be provided as a removable component (such as a removable film, foil or laminate) that encloses the membrane and prevents evaporation of the volatile composition before the sealing substrate is removed. When a sealing substrate of this type is removed from the cartridge, the entire membrane may be exposed to a surrounding environment, enabling an increased evaporation rate of volatile composition. In such cases, the sealing substrate may be provided with a pull tab to allow for easy removal of the sealing substrate from cartridge prior to use by a consumer. Therefore, in some configurations, the sealing substrate encloses both the membrane and reservoir, and the sealing substrate is configured to be removed before use.

Where the sealing substrate is provided with a pull tab to facilitate removal of the scaling substrate, the pull tab may be any suitable size. For example, the pull tab may have a length of from about 20 mm or less, preferably about 15 mm or less, or more preferably about 10 mm or less. As another example, the pull tab may have a length of from about 2 mm to about 20 mm, preferably from about 5 mm to about 15 mm, or more preferably from about 5 mm to about 10 mm, specifically including all values within these ranges and any ranges created thereby. Similarly, the width of the pull tab may be about 20 mm or less, preferably about 15 mm or less or more preferably about 10 mm or less. As another example, the pull tab may have a width of from about 2 mm to about 20 mm, preferably from about 5 mm to about 15 mm, or more preferably from about 5 mm to about 10 mm, specifically including all values within these ranges and any ranges created thereby. The pull tab may be positioned in any suitable location on the sealing substrate. Preferably the pull tab is disposed adjacent the seal area.

Where the length and/or width of the pull tab exceeds the above, the cartridges may be difficult to package. For example, the housing and the cartridge may be packaged together or separately. Where the cartridge is disposed within the housing, the housing may be in the open position or the closed position. When in the closed position, it would be desirable to ensure the pull tab is similarly disposed within the housing as well. Where the cartridge is disposed outside of the housing, it may be useful to show a portion of the pull tab to a consumer to demonstrate the facility with which the cartridges may be activated; however, where the length and/or width exceed the above values, it may be difficult to show only a portion while hiding the remainder of the pull tab.

It is worth noting that where separation slots are provided in the housing, the pull tab may be configured such that the pull tab extends through the separation slot. In such configurations, the user may remove the sealing substrate through the separation slot. Additionally, in order to facilitate removal of the sealing substrate, the separation slot may be appropriately sized to allow the sealing substrate to pass through the separation slot without substantial deformation and additional force required by the user.

As described, in some configurations, the sealing substrate may be a rupturable substrate. In other configurations, the sealing substrate may be a removable scaling substrate.

When the sealing substrate is ruptured during closing of the housing as described herein, a membrane may become wetted with the volatile composition. Since the volatile composition typically comprises various organic compounds and solvents, users typically prefer to avoid needing to touch a wetted membrane. The configuration described herein advantageously allows the cartridge to be activated without a user needing to touch the cartridge during the activation step, thereby avoiding any contact between a user's hands and organic components of the volatile composition. Therefore, in some configurations described herein, the scaling substrate is configured to be ruptured during closing of the housing.

### Compressive Force

As described herein, the rear frame may comprise one or more rib elements, which are configured to apply a compressive force on the cartridge. An opposing side of the cartridge is held in place by the front frame, such that the compressive force is generated by the compression of the cartridge between the front frame and the rib elements. While the compressive force is generated by compression between the front frame and rib elements, for the sake of brevity and simplicity, the compressive force is described herein in relation to the action of the rib elements on the cartridge.

Typically, the rib elements are configured to apply the compressive force on the membrane of a cartridge when the housing is in a closed state, either through direct impact of the rib elements on the membrane, or through one or more additional components or layers. The compressive force may be applied to the cartridge for substantially the entire lifetime of the cartridge while it is positioned within the housing. In other words, the compressive force may be applied to the cartridge for substantially as long as the cartridge is present within the housing and volatile composition is evaporating from the cartridge. In this context, the term substantially may mean at least 80% of the time, at least 85% of the time, at least 90% of the time, or at least 95% of the time.

As used herein, a compressive force refers to a force that acts to compress a part of the cartridge, and may be understood as a force that acts on a surface of the cartridge, in a direction towards an interior of the cartridge. The compressive force may act substantially perpendicular to a surface of the cartridge, but a person skilled in the art will understand that the compressive force may act at an angle relative to a surface of the cartridge and still provide the benefits discussed below. The compressive force may typically act at a direction that is substantially perpendicular to a surface of the cartridge, such as from 60° to 120° to a surface of the cartridge.

The compressive force may have any appropriate magnitude. The magnitude of the compressive force should be high enough that the compressive force is able to achieve the effects described below, but should not be excessively high such that it undesirably ruptures or otherwise damages the cartridge. Thus, the magnitude of the compressive force may be, for example, from about 10 N to about 140 N, such as about 20 N to about 120 N. Nevertheless, other compressive forces may also be appropriate in some circumstances, such as 10 N to 20 N, 10 N to 30 N, 10 N to 40 N, 10 N to 50 N, 10 N to 60 N, 10 N to 70 N, 10 N to 80 N, 10 N to 90 N, 10 N to 100 N, 10 N to 110 N, 10 N to 120 N, or 10 N to 130 N. For the avoidance of doubt, any end point of any range disclosed herein may be combined with any other end point of any range disclosed herein in respect of the same variable. For example, for the above ranges of compressive force, the following are to be considered disclosed:
20 N to 30 N, 20 N to 40 N, 20 N to 50 N, 20 N to 60 N, 20 N to 70 N, 20 N to 80 N, 20 N to 90 N, 20 N to 100 N, 20 N to 110 N, 20 N to 120 N, or 20 N to 130 N,
30 N to 40 N, 30 N to 50 N, 30 N to 60 N, 30 N to 70 N, 30 N to 80 N, 30 N to 90 N, 30 N to 100 N, 30 N to 110 N, 30 N to 120 N, or 30 N to 130 N,
40 N to 50 N, 40 N to 60 N, 40 N to 70 N, 40 N to 80 N, 40 N to 90 N, 40 N to 100 N, 40 N to 110 N, 40 N to 120 N, or 40 N to 130 N,
50 N to 60 N, 50 N to 70 N, 50 N to 80 N, 50 N to 90 N, 50 N to 100 N, 50 N to 110 N, 50 N to 120 N, or 50 N to 130 N,
60 N to 70 N, 60 N to 80 N, 60 N to 90 N, 60 N to 100 N, 60 N to 110 N, 60 N to 120 N, or 60 N to 130 N,
70 N to 80 N, 70 N to 90 N, 70 N to 100 N, 70 N to 110 N, 70 N to 120 N, or 70 N to 130 N,
80 N to 90 N, 80 N to 100 N, 80 N to 110 N, 80 N to 120 N, or 80 N to 130 N,
90 N to 100 N, 90 N to 110 N, 90 N to 120 N, or 90 N to 130 N,
100 N to 110 N, 100 N to 120 N, or 100 N to 130 N,
110 N to 120 N, or 110 N to 130 N,
120 N to 130 N.

A person skilled in the art will appreciate that smaller or greater compressive forces may be suitable in some circumstances. For example, in some configurations the compressive force may be as low as 3 N, such as from 3 N to 10 N.

In some preferred configurations, the compressive force may have a magnitude of from about 30 N to about 120 N, such as from about 35 N to about 100 N, such as from about 40 N to about 90 N.

The compressive force may be approximated as equal to the opening force described below, and for the purposes of the present disclosure, the magnitude of the compressive force may be measured by measuring the opening force as described herein.

This compressive force provides several benefits that are described below.

First, the force applied by the rib elements may cause rupture of the sealing substrate when the housing is closed, as described in more detail herein (e.g. by actuating a rupture mechanism located underneath the membrane). In this case, the compressive force causes activation of the cartridge simply by closing the housing, avoiding a common problem with the prior art involving push buttons or other activation mechanisms, where a user is unsure whether or not they have applied sufficient force on the button to properly activate a cartridge.

Second, the compressive force applied by the rib elements on the cartridge causes an equal and opposite reaction force applied by the cartridge on the housing, which reaction force biases opening of the housing. This reaction force may be referred to herein as an “opening force”. Typically, the housing comprises a locking structure for releasably locking the housing in a closed state, such that the opening force does not cause the housing to open while the locking structure is engaged. However, when the locking structure is released or disengaged, the housing will at least partially spring open due to the opening force applied on the housing by the cartridge held within the housing. Thus, in some configurations described herein, when the housing is in a closed state, the cartridge applies an opening force on the housing, where the term “opening force” is to be understood as a force that biases opening of the housing. This advantageously increases the case of opening the housing, because when the locking structure is disengaged or unlocked, the housing will at least partially spring open, allowing for an easy grip of the front and rear frames. The opening force may have a magnitude that is equal to the magnitude of the compressive force described above. In some configurations, the opening force may have a magnitude of from about 3 N to about 140 N, from about 10 N to about 100 N, from about 20 N to about 60 N, preferably from about 25 N to about 45 N. The opening force may be measured by closing the housing with a new cartridge, securing the front or rear frame at an angle such that a seam between the front and rear frames is parallel to the horizontal and measuring the peak vertical opening force at a midpoint of the housing when the locking structure is released/disengaged (taking the force of gravity into account).

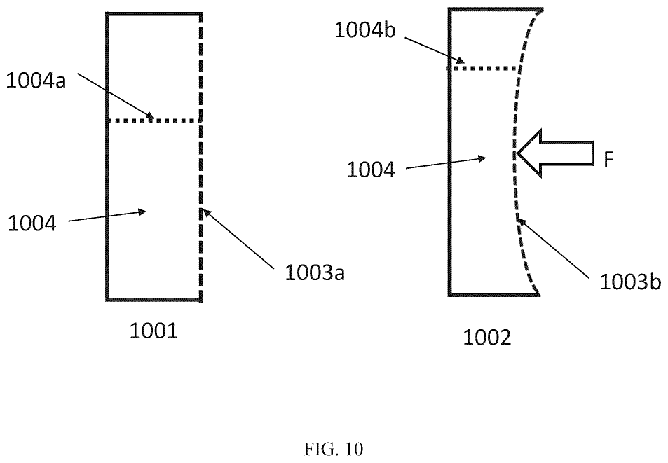

Third, the cartridge includes a reservoir containing a volatile composition. When the compressive force acts on a deformable part of the reservoir, or the membrane enclosing the reservoir, a resulting deformation may decrease the thickness of the reservoir in the direction the force acts along. Typically, the compressive force acts in a direction that is substantially perpendicular to a plane of the membrane, such as from 60° to 120° to the membrane. When this direction is a horizontal direction, which is the case when the volatile composition dispenser is in its intended upright configuration as shown in FIG. 6A, the reduced horizontal thickness of the reservoir causes an increase in a height of the liquid volatile composition within the reservoir. As shown in FIG. 10, this provides the appearance of a higher fill level, increasing consumer satisfaction with the volatile composition dispenser, especially when a fill level of volatile composition is easily visible through a window portion of the front frame of the housing.

Fourth, when the compressive force acts on a deformable part of the reservoir, or the membrane enclosing the reservoir, the resulting deformation ensures that the cartridge is held tightly within the housing and does not move or rattle within the housing when the housing is moved. This provides a premium feel to the product, increasing user satisfaction. The effect of this benefit may be increased when the front frame of the housing comprises a window portion configured to receive the reservoir of the cartridge, as described herein.

As noted, the cartridge and housing may be packaged together, and the cartridge may be disposed within the housing in the package. In such configurations, where the housing is closed and the cartridge is configured to be activated by the rib elements, additional sealing measures should be taken to ensure that the volatile composition does not evaporate prior to intended use by a consumer. In another packaging configuration, the housing may be in the open or closed position while the cartridge is spaced from the housing within the same package. In yet another configuration, the housing may be sold in a separate package from the cartridge. In any of the foregoing configurations, more than one cartridge may be included in the packaging and such cartridges may have the same volatile composition or may comprise a different volatile composition.

### Volatile Composition

The volatile composition can comprise, but is not limited to, a substance that can function as an air freshener, a deodorant, an odor neutralizing material, an odor blocking material, a malodor counteractant, an odor masking material, an aromatherapy material, an aromachology material, an insecticide, air and/or surface sanitizer, and/or a combination thereof. In other various configurations, the volatile composition can comprise other various materials that can act in their vapor phase to modify, enhance, and/or treat an atmosphere or an area outside of the volatile composition dispenser.

The term “volatile composition” as used herein, refers to a material that is vaporizable at room temperature and atmospheric pressure without the need of an energy source. The volatile composition may be a composition comprised entirely of a single volatile material. The volatile composition may also be a composition comprised entirely of a volatile material mixture (i.e. the mixture has more than one volatile component). Further, it is not necessary for all of the component materials of the composition to be volatile. Any suitable volatile composition in any amount or form, including a liquid or emulsion, may be used.

Liquid suitable for use herein may, thus, also have non-volatile components, such as carrier materials (e.g., water, solvents, etc). It should also be understood that when the liquid is described herein as being “delivered”, “emitted”, or “released,” this refers to the volatilization of the volatile component thereof, and does not require that the non-volatile components thereof be emitted.

The volatile composition can be in the form of perfume oil. Most conventional fragrance materials are volatile essential oils. The volatile composition can be a volatile organic compound commonly available from perfumery suppliers. Furthermore, the volatile composition can be synthetically or naturally formed materials. Examples include, but are not limited to: oil of bergamot, bitter orange, lemon, mandarin, caraway, cedar leaf, clove leaf, cedar wood, geranium, lavender, orange, origanum, petitgrain, white cedar, patchouli, neroili, rose absolute, and the like. In the case of air freshener or fragrances, the different volatile materials can be similar, related, complementary, or contrasting.

It may be desirable for the volatile composition to be in the form of a liquid at 25° C. As explained herein, the microporous membranes used in the examples of the present disclosure may have advantageously increased visual appearance changes when wetted with volatile composition.

The volatile composition may have a combined vapour pressure of at least 8 Pa at 25° C., such as at least 30 Pa at 25° C.

In addition to, or as part of, the volatile composition, the cartridge may include any known malodor composition to neutralize odors. Suitable malodor compositions include cyclodextrin, reactive aldehydes and ionones.

While not wishing to be bound by theory, the continuous delivery of a volatile composition may be a function of various factors including membrane pore size; membrane surface area; the physical properties of a volatile composition, such as molecular weight and saturation vapor pressure (“VP”); and the viscosity and/or surface tension of the volatile composition.

The volatile composition may be formulated such that the composition comprises a volatile material mixture comprising about 10% to about 100%, by total weight, of volatile materials that each having a VP at 25° C. of less than about 0.01 torr; alternatively about 40% to about 100%, by total weight, of volatile materials each having a VP at 25° C. of less than about 0.1 torr; alternatively about 50% to about 100%, by total weight, of volatile materials each having a VP at 25° C. of less than about 0.1 torr; alternatively about 90% to about 100%, by total weight, of volatile materials each having a VP at 25° C. of less than about 0.3 torr. In one example, the volatile material mixture may include 0% to about 15%, by total weight, of volatile materials each having a VP at 25° C. of about 0.004 torr to about 0.035 torr; and 0% to about 25%, by total weight, of volatile materials each having a VP at 25° C. of about 0.1 torr to about 0.325 torr; and about 65% to about 100%, by total weight, of volatile materials each having a VP at 25° C. of about 0.035 torr to about 0.1 torr. One source for obtaining the saturation vapor pressure of a volatile material is EPI Suite™, version 4.0, available from U.S. Environmental Protection Agency.

Two exemplary volatile compositions comprising a volatile material mixture having volatile materials of varying VPs are set forth below in Tables 1 and 2. These compositions arc shown by way of illustration and are not intended to be in any way limiting to the scope of the present disclosure.

TABLE 1

| Wt % | Low VP (torr) | High VP (torr) |
|---|---|---|
| 27.71 | 0.175 | 0.325 |
| 20.78 | 0.0875 | 0.1125 |
| 13.86 | 0.0625 | 0.0875 |
| 8.66 | 0.0375 | 0.0625 |
| 8.66 | 0.0175 | 0.0325 |
| 6.93 | 0.00875 | 0.01125 |
| 6.93 | 0.00625 | 0.00875 |
| 3.18 | 0.00375 | 0.00625 |
| 1.27 | 0.00175 | 0.00325 |
| 0.95 | 0.000875 | 0.001125 |
| 0.64 | 0.000625 | 0.000875 |
| 0.32 | 0.000375 | 0.000625 |
| 0.09 | 0.000175 | 0.000325 |

TABLE 2

| Wt % | Low VP (torr) | High VP (torr) |
|---|---|---|
| 33.38 | 0.175 | 0.325 |
| 25.75 | 0.0875 | 0.1126 |
| 19.07 | 0.0625 | 0.0875 |
| 13.86 | 0.0375 | 0.0625 |
| 4.00 | 0.0175 | 0.0325 |
| 1.50 | 0.00875 | 0.01125 |
| 0.50 | 0.00625 | 0.00875 |
| 0.72 | 0.00375 | 0.00625 |
| 0.55 | 0.00175 | 0.00325 |
| 0.27 | 0.000875 | 0.001125 |
| 0.20 | 0.000625 | 0.000875 |
| 0.13 | 0.000375 | 0.000625 |
| 0.07 | 0.000175 | 0.000325 |

The viscosity of a volatile composition may control how and when a volatile composition is delivered to the microporous membrane. For example, less viscous compositions may flow faster than the more viscous volatile compositions. Thus, the membrane may be first wetted with the less viscous materials. To help prevent liquid from seeping through the microporous membrane, volatile compositions may have viscosities less than about 23 cP and surface tension less than about 33 mN/m.

In one example, the volatile composition may have a viscosity of about 1.0 cP to less than about 25 cP, alternatively about 1.0 cP to less than about 23, alternatively about 1.0 cP to less than about 15 cP.

The volatile composition may be designed such that the composition may include a surface tension of about 19 mN/m to less than about 33 mN/m, alternatively about 19 mN/m to less than about 30 mN/m, alternatively about 19 mN/m to less than about 27 mN/m.

The present disclosure also provides a method of delivering a volatile composition to an interior space, comprising the steps of:
(i) providing a volatile composition dispenser as defined herein in an interior space; and
(ii) activating the volatile composition dispenser to enable release of the at least one liquid volatile composition.

### Consumer Product

Cartridges for the volatile composition dispenser may be provided in the form of a consumer product. Thus, the present disclosure also provides a consumer product comprising:
(i) an outer packaging;
(ii) one or more cartridges as defined herein; and
(iii) instructions for using the one or more cartridges with a reusable housing as defined herein, wherein the one or more cartridges are packaged within the outer packaging.

In some configurations, the consumer product may take the form of a refill pack of cartridges, where the refill package does not comprise a reusable housing referred to in the instructions. The components of the consumer product are discussed below.

The outer packaging may be any appropriate packaging, such as a rigid or soft packaging. From the perspective of protecting the cartridges against accidental damage or rupture, the packaging may be rigid or semi-rigid. For example, the packaging may be formed from a plastics material, or from a cardboard material. In some configurations, the outer packaging may be formed from a combination of rigid and semi-rigid materials, such as a combination of plastics materials and cardboard materials.

In some configurations, the outer packaging may be formed from a majority of cardboard materials. For example, the outer packaging may be formed from at least 50 wt. % cardboard materials, such as at least 60 wt. %, at least 70 wt. %, at least 80 wt. %, at least 90 wt. %, at least 95 wt. % cardboard materials.

The outer packaging may take any appropriate form. In some configurations, the outer packaging may take the form of a blister or clam-shell packaging including a combination of plastics and cardboard. A blister packaging may have any appropriate construction, including a face seal blister, full-face seal blister, full card blister, and trapped blister. In some configurations, the outer packaging may take the form of a box, such as a cardboard box.

Suitable cardboard materials that may be utilized in the outer packaging include corrugated cardboard (such as single face, single wall, double wall, triple wall and honeycomb corrugated cardboard) and paperboard. In some configurations, the cardboard materials may be laminated with a plastics material.

Suitable plastics materials that may be utilized in the outer packaging include PET (polyethylene terephthalate) and PVC (polyvinyl chloride), though a skilled person will appreciate that other plastics may be used.

The consumer product comprises instructions for using the one or more cartridges with a reusable housing. In this context, “using” may be understood as placing the cartridge into the reusable housing in such a way that the reusable housing supports the cartridge and enables an effective release of volatile material without causing spillage or leakage of the volatile material. The use of the cartridge with a housing as specified in the instructions may also provide additional advantages discussed herein, which advantages may not be obtained if the cartridge is used with a housing that is not as specified in the instructions. These advantages are discussed in detail herein with respect to the housing.

While the instructions may be provided within the outer packaging, e.g. as an insert within the outer packaging, or as a marking on the one or more cartridges themselves, it is not necessary for the instructions to be physically packaged within the outer packaging. The instructions may be provided in any format such that they are provided to the consumer at the point or purchase or use of the consumer product. Thus, it is explicitly contemplated herein that the instructions may be present on the exterior of the outer packaging, alongside the outer packaging as a separate component (e.g. on a shelf alongside the consumer product) or provided electronically during use or purchase (e.g. as part of an advertisement or sales listing accessed electronically).

The instructions for use instruct a user to use the one or more cartridges with a reusable housing, for which the cartridges are adapted. The instructions may take the form of a written note or graphical indication that the one or more cartridges are suitable for, or compatible with, a specific reusable housing. Such a note or indication is taken to constitute instructions that direct a user to place the one or more cartridges into the mentioned housing, which may typically have features defined hereinbelow. Therefore, in some configurations examples of the present disclosure provide a consumer product wherein the instructions direct a user to use the one or more cartridges with a reusable housing that has any combination of features defined herein.

The instructions may direct a user to place the one or more cartridges into a single specific reusable housing, such as a specific branded housing, where such a housing has properties defined herein. Alternatively, the instructions may provide a list of possible reusable housings that the one or more cartridges may be used with, where each of the listed housings have properties defined herein.

Various configurations will now be described to provide an overall understanding of the principles of the structure, function, manufacture, and use of the apparatuses and methods disclosed herein. One or more examples of these configurations are illustrated in the accompanying drawings. Those of ordinary skill in the art will understand that the apparatuses and methods specifically described herein and illustrated in the accompanying drawings are non-limiting example configurations and that the scope of the various configurations of the present disclosure are defined solely by the claims. The features illustrated or described in connection with one example configuration may be combined with the features of other example configurations. Such modifications and variations are intended to be included within the scope of the present disclosure.

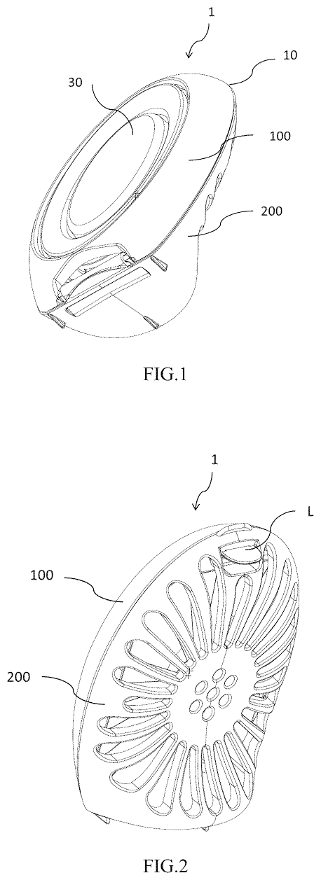

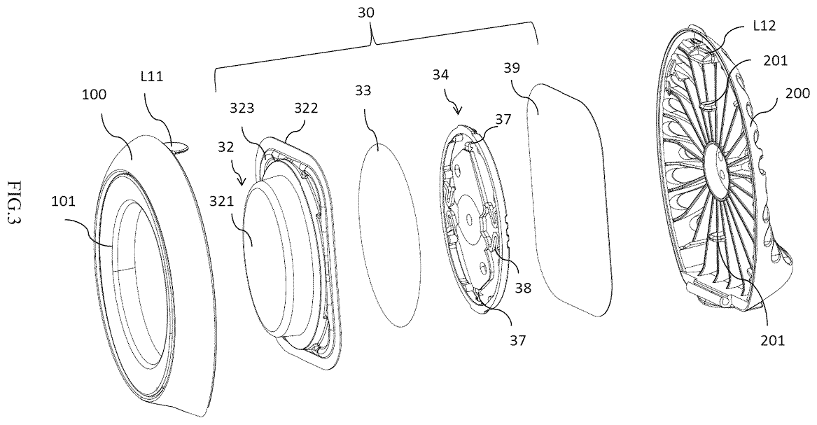

FIG. 1 shows a front perspective view of an example of a volatile composition dispenser 1 (hereinafter “dispenser”) in a closed state and FIG. 2 shows a rear perspective view of the dispenser 1. The dispenser 1 comprises a housing 10 having a front frame 100 and a rear frame 200. The front frame 100 and the rear frame 200 are configured to be capable of moving away from or toward each other for opening and closing the housing. The front frame 100 and the rear frame 200 may be completely detachable from each other and capable of moving toward and away from each other without any restraint, or may be connected at a first end but be capable of moving away from or toward each other at the opposing second end. For example, the front frame 100 and the rear frame 200 may be connected at a first end and are capable of pivotally moving away from or toward each other at the opposing second end for opening and closing the housing. When the housing 10 is closed, the front frame 100 and the rear frame 200 define an interior space. A cartridge 30 containing a volatile composition is located within the interior space of the housing 10. FIG. 2 also shows a locking structure L (L1) which can releasably interlock the front frame 100 and the rear frame 200 together upon closing of the housing 10. The details of the locking structure L will be described later.

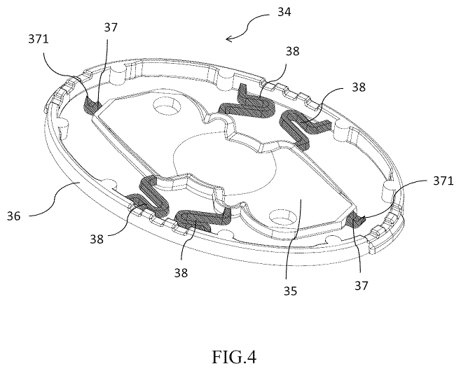

FIG. 3 shows internal components of a dispenser 1, which comprises a rupture mechanism. The front frame 100 comprises a window portion 101 configured for displaying the cartridge 30. The window portion 101 may be configured in various different shapes, including, for example, circle, oval, rectangle, square, and the like.

The cartridge 30 can be configured in a variety of ways. The cartridge may contain a liquid volatile composition that is configured to evaporate through a membrane. With reference to FIG. 3, the cartridge 30 comprises a container 32 having a reservoir 321, within which the volatile composition is stored. A sealing substrate 33 is sealably attached to and covers the reservoir 321 to prevent the volatile composition from being released until the dispenser 1 is activated. The sealing substrate 33 may be ruptured to release the volatile composition 31 by actuating a rupture mechanism 34 positioned adjacent to the sealing substrate 33. One or more rupture elements 37 are arranged within the rupture mechanism 34 to puncture holes in the sealing substrate 33. The rupture element 37 may be a pin. The cartridge 30 may comprise a membrane 39 located on the exterior of the cartridge 30. The membrane 39 may be sealably attached to a flange 322 located at a periphery 323 of the container 32. The membrane 39 may enclose the container 32, the volatile composition, the sealing substrate 33, and the rupture mechanism 34. The membrane 39 may be configured to flex when a pressure or an actuation force is applied on the membrane 39. And the membrane 39 may be configured to porous so as to sustainably absorb and volatilize the volatile composition.

As noted, in some configurations, the cartridge may comprise a removable sealing substrate located externally to the membrane. In such configurations, the cartridge may not require a rupture mechanism to rupture the scaling substrate.

A shape of the container 32/reservoir 321 may be configured to correspond to a shape of the window portion 101 of the front frame 100. For example, the container 32/reservoir 321 may define a substantially elliptical or oval shape and its width to length ratio may be about 1:2 to 1:2.5. By allowing the container 32/reservoir 321 to fit into the window portion 101 of the front frame 100, the cartridge 30 can be positioned in place, not be moving around inside the front frame.

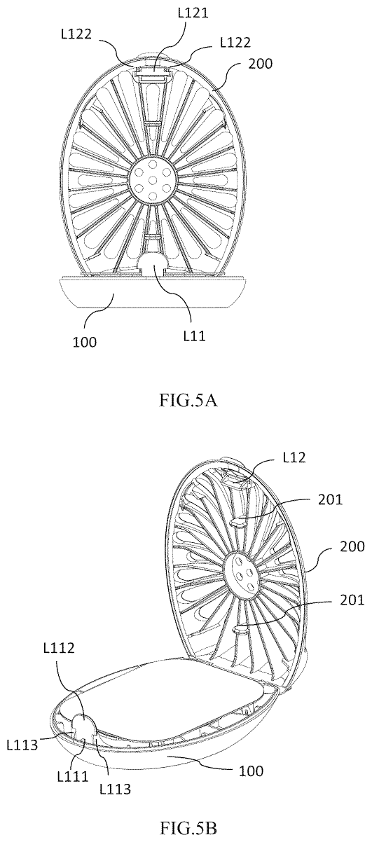

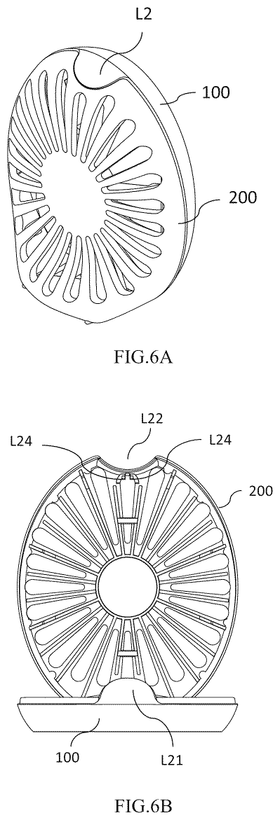

The rupture mechanism 34 may comprise an outer frame 36 and a movable member 35 movably attached to the outer frame 36 by a resilient member 38, as shown in FIG. 4. The resilient member 38 may be formed of one or more springs. The rupture mechanism 34 further comprises a plurality of rupture elements 37 arranged on the edges of the movable member 35. As shown in FIG. 4, the rupture elements 37 are spaced apart along the periphery of the rupture mechanism 34 and extend from at least two sides orthogonal to the surface of the rupture mechanism, wherein each rupture element 37 comprises a tip 371 for puncturing the sealing substrate 33. The rupture elements 37 are generally disposed in pairs. For example, a pair of rupture elements 37 disposed on opposing sides of the movable member 35 enable puncturing at least a pair of opposing holes in the sealing substrate 33, a hole at one side of the sealing substrate 33 allowing air to enter the container 32, and another hole at an opposing side draining the volatile composition from the container 32 so as to impregnate the membrane 39.

The rear frame 200 comprises one or more rib elements 201 configured for applying a compressive force on the cartridge 30 upon closing the housing 10 when the front and rear frames move toward each other. This compressive force provides a number of benefits as discussed hereinabove. The rear frame 200 may comprise a plurality of rib elements 201 configured to be spatially aligned with the rupture elements 37 of the rupture mechanism 34, so that when the rib elements 201 come into contact with the cartridge 30, the rib elements press onto the rupture elements 37 and thereby causing rupture of the sealing substrate 33. Similar to the arrangement of rupture elements 37, the rib elements 201 may be disposed in pairs near periphery of the rear frame 200. For example, a pair of rib elements 201 may be disposed on first opposing sides of the rear frame (e.g. in a vertical orientation), as shown in FIG. 3.

Alternatively, the rib elements 201 may be configured as rupture elements, each comprising a tip for puncturing the sealing substrate 33 via the membrane 39 when closing the housing 10. In such case, the rupture mechanism 34 in the cartridge 30 can be omitted. However, due to the cushion of the membrane 39, it requires more force upon closing of the housing to cause the rupture of the sealing substrate 33 (in some cases, the consumer may have to open and close the housing repeatedly, to actuate the cartridge 30 (by puncturing the sealing substrate 33) properly).

The rib elements 201 may have a ridge shape, protruding from the internal surface of the rear frame 200, and extending from the periphery of the rear frame 200. The peripheral arrangement of the rib elements 201 is more preferable, because it ensures accurate localization of the compressive force upon the rupture elements 37 by aligning the rib elements 201 with the rupture elements 37. The rib elements 201 may extend along a perpendicular direction in relation to the plane defined by the rear frame, or along a slanted direction (i.e., neither parallel nor perpendicular) in relation the plane defined by the rear frame. But in any case, it is preferable that the rib elements 201 are orthogonal to the plane defined by the front frame when the housing is closed completely, so that the rib elements can apply a maximum compressive force on the cartridge 30.

The rib element 201 may have a height of from 0.1 to 1 cm, so that it may cause sufficient deformation of (the membrane 39 of) the cartridge 30 to actuate the rupture elements 37 but do not cause great hindrance when closing the housing 10. For similar reasons, the rib element 201 may have an extending length ranges from 0.1 to 2 cm.

The rib element 201 may comprises a cartridge-contacting surface away from the internal surface of the rear frame 200. The cartridge-contacting surface may have a width (i.e. the width of the rib element 201) ranges from 0.1 to 0.5 cm. The cartridge-contacting surface may be flat or rounded, and is preferably substantially free of protrusions, so that the contact area between the rib element 201 and the cartridge 30 is large enough to apply large compressive force evenly.

The front frame 100 and the rear frame 200 are configured to be capable of moving away from or toward each other for opening and closing the housing. In a configuration, the front frame 100 and the rear frame 200 may be connected at a first end and are capable of pivotally moving away from or toward each other at the opposing second end for opening and closing the housing. Optionally, the front frame 100 and the rear frame 200 are connected at the first end through a hinge structure, which comprises a knuckle element located on either of the front and rear frames and a pin element located on the other of the front and rear frames. The knuckle element or the pin element may be molded as an integral part of the front or rear frame.

The front frame 100 and the rear frame 200 may further comprise a locking structure to releasably interlock the front and rear frames together upon closing of the housing 10, as shown in FIGS. 2 and 6A. The locking structure may be disposed at the movable end (e.g. the second end) of the housing 10. Various locking structures can be utilized, including, for example, latches, snap fits, clasps and catches. Representative latch locking structures include through-latch and button latch structures.

FIGS. SA to 5B show the configuration of a through-latch locking structure L1. The through-latch locking structure L1 fulfills the locking function by the cooperation of a door latch portion L11 located on one of the front and rear frames and a through-hole portion L12 located on the other of the front and rear frames. Typically, the door latch portion L11 is a slat-like element having a proximal end L111 fixed on one of the front and rear frames and a distal end L112 extending outwardly (e.g. toward the other one of the front and rear frames). The through-hole portion L12, located on the other one of the front and rear frames, comprises a hole or a slit L121 penetrating the entire thickness of the frame and having a dimension allowing the distal end L112 of the door latch portion L11 to pass through, and further comprises an overhang member L122 hanging from the wall of the hole or slit L121 and extending inwardly so that it can be engaged with the door latch portion L11 when the distal end L112 passes through the hole or slit L121, thereby locking the through-latch structure. When applying an external force, the overhang member L122 can be disengaged with the door latch portion L11, allowing the distal end L112 to be withdrawn from the hole or slit L121, thereby unlocking the through-latch structure.

For example, the door latch portion L11 may comprise one or more notches L113 in the section adjacent to the distal end L112. The notch may be a complete internal hole (e.g. at the center of the section in the width direction), or a semi-open hole at the edge of the section. When the distal end of the door latch portion passes through the hole or slit of the through-hole portion, the overhang member L122 may insert into the notch L113, so that the door latch portion L11 is locked.

The door latch portion L11 is configured to flex so that it can be deflected in the thickness direction when an external force is applied. The overhang member L122 may also be flexible and can be deformed by applying a force thereon. Thus, the door latch portion L11 can be unlocked by applying a force to (the distal end of) the door latch portion in the thickness direction thereof, deflecting the door latch portion to allow the overhang member L122 detach from the notch L113. Similarly, the overhang member L122 can be subjected to a deformation by applying a force thereon, and subsequently be released from the notch L113. Of course, both the door latch portion and the overhang member can be applied a force at the same time, to detach them from each other.

Alternatively, the locking structure may take the form of a button latch L2 that comprises an overhang button L21 and a latch member L23 and utilizes the cooperation of these two members to lock and release the device. FIG. 6A shows a configuration of a volatile composition dispenser in a locked state, which comprises a button latch L2. The button latch L2 comprises an overhang button L21 with flexibility, which is located at an edge of the front frame 100, and a complementary groove L22 at the corresponding edge of the rear frame 200. When the housing 10 is closed and the front and rear frames are locked, the overhang button L21 can just fill the groove L22, forming a smooth and complete appearance.

FIG. 6B-6C show the housing 10 in fully opened state. The button latch L2 further comprises a latch member L23 on the inner side of the button L21, and the latch member L23 comprises a latch body L233 and a pillar L231 for connecting the button L21 and the latch body L233. Two semi-open notches L232 (between the button L21 and the latch body L233) are formed by the button L21, the pillar L231 and latch body L233. Two protrusions L24 are disposed near the groove L22, extending from the inner surface of the rear frame 200, at positions corresponding to the notches L232. When the housing 10 is closed, the protrusions L24 enter the notches L232, and at the same time, the pillar L231 is stuck in the gap between the protrusions L24, providing a stable lock between the front frame 100 and the rear frame 200. In order to open the housing 10 again, the consumer may press the button L21, to make deformation of the button, so that the pillar L231 breaks away from the gap and the protrusions L24 detach from the notches, thereby unlocking the latch structure.

The overhang button L21 may be located at the edge of the front frame 100 and the groove L22 may be located at the corresponding edge of the rear frame 200, as shown by FIGS. 6A-6C, and it is possible that the overhang button L21 may be located at the edge of the rear frame 200, and correspondingly, the groove L22 may be located at the front frame 100. Furthermore, the button latch L2 may be disposed at a position different from the top side as shown in FIGS. 6A-6C, for example, it can be located at the left or right side or both of the housings 10.

One of the advantages of the button latch structure is to provide an aesthetic appearance. In addition, the larger button area provides the consumer's comfort when unlocking the locking structure.

The volatile composition volatile composition dispenser can be actuated by placing the cartridge 30 inside the front frame 100, with the container 32 fitting into the window portion 101 of the front frame 100, and then moving the front frame 100 and the rear frame 200 toward each other to bring the rib elements 201 into contact with the cartridge 30 to cause rupture of the sealing substrate 33, and optionally, releasably interlocking the front frame 100 and the rear frame 200.

When the cartridge 30 is depleted of volatile composition, a user can unlock the locking structure to open the housing 10, remove the depleted cartridge away from the housing, and re-fill another cartridge 30 inside the housing 10, followed by closing of the housing 10.

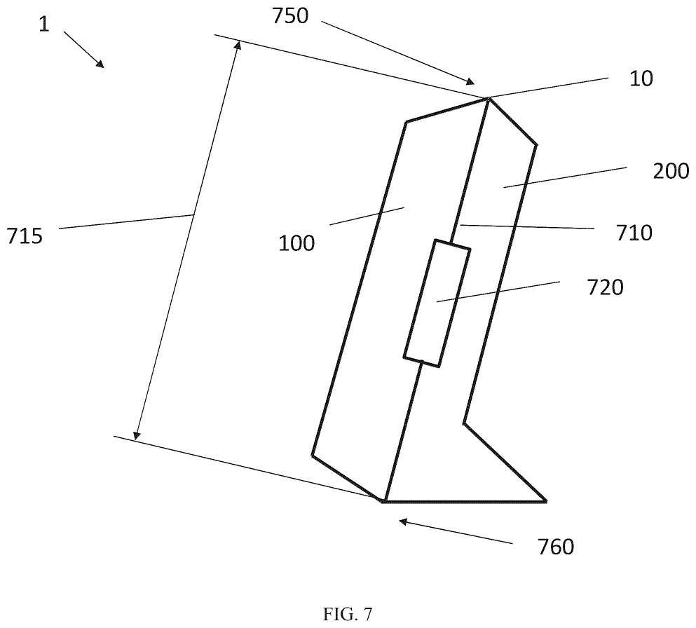

As shown in FIG. 7, the housing 10 may comprise a separation slot 720 to facilitate separation of the front frame 100 from the rear frame 200. As shown, the separation slot 720 may be positioned at an interface 710 between the front frame 100 and the rear frame 200. An additional separation slot may be provided on an opposite side of the housing 10. It is worth noting that one or more separation slots 710 may be positioned opposite a pivot axis. For example, a separation slot 710 may be provided at an upper portion 750 of the housing where a pivot axis is located at a lower portion 760 of the housing 10. Conversely, a separation slot 710 may be provided at the lower portion 760 where a pivot axis is located at an upper portion 750.

Where it is desired to remove the sealing substrate 33 (shown in FIG. 3) from the cartridge 30 (shown in FIG. 3), the separation slot 720 or an opposing separation slot may have a length (generally parallel to a length 715 of the interface 710, which is at least 60 percent of the length 715 of the interface 710, more preferably at least 70 percent, even more preferably at least 80 percent of the length 715 of the interface 710. Regardless of whether the sealing substrate will be removed from the separation slot 710, the separation slot 710 may comprise a length which is at least about 10 percent of the length 715 of the interface 710. For example, the separation slot 710 may comprise a length which is from about 10 percent to about 60 percent, more preferably from about 10 percent to about 70 percent, or even more preferably from about 10 percent to about 80 percent of the length 715 of the interface 710.

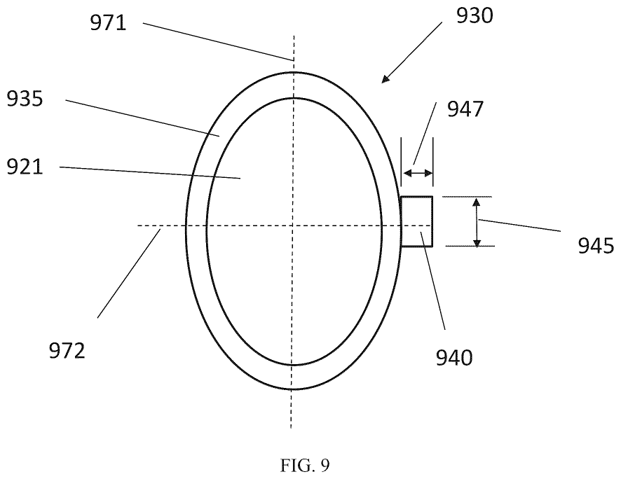

Referring now to FIGS. 7 and 9, wherein the separation slots are disposed opposite a pivot axis of the housing 10, i.e. separation slot in the upper portion 750 or lower portion 760, the separation slot may comprise a length generally parallel to a minor axis 972 of the cartridge 930. The values provided above regarding the length of the separation slot 720 apply in this configuration as well. It is worth noting that in such configurations, the length of the separation slot 720 may be generally parallel to the minor axis 972 of the cartridge 930.

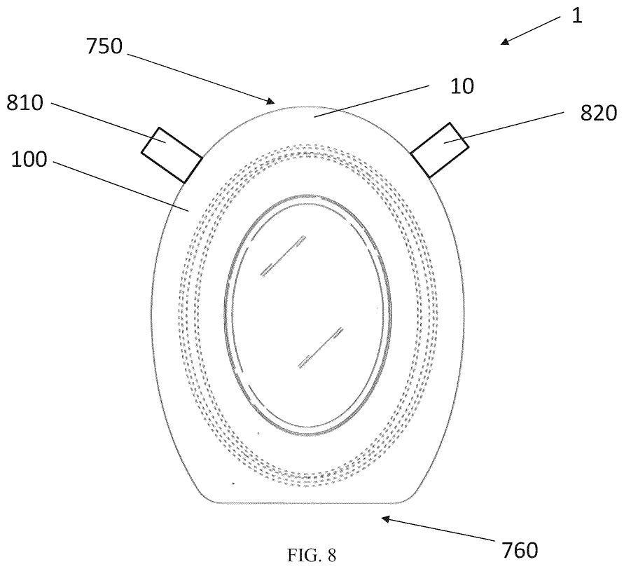

Referring now to FIGS. 7 and 8, in combination with the separation slots or independently thereof, the housing 10 may comprise separation tabs 810 and 820. The separation tabs 810 and 820 may be comprised by the front frame 100, the rear frame 200 or one each by the front frame 100 and the rear frame 200. The configuration shown in FIG. 8 may be beneficial where the front frame 100 and the rear frame 200 are pivotable with respect to one another, particularly where a pivot axis is located at lower portion 760 of the housing 10. In contrast, the separation tabs 810 and 820 may be positioned adjacent the lower portion 760 of the housing 10 where the pivot axis is located at an upper portion 750 of the housing 10. Additional separation tabs may be provided on the front frame 100 and/or rear frame 200. In such configurations, the additional separation tabs may be spaced apart from the separation tabs 810 and 820. The additional separation tabs may be beneficial where the front frame 100 is completely separable from the rear frame 200.

Referring now to FIG. 9, a cartridge 930 suitable for use in conjunction with the housing 10 in accordance with the present description is shown. The cartridge 930 comprises a seal area 935 which, as noted herein may comprise a portion of the sealing substrate 921 and a portion of the membrane. In order to facilitate removal of the sealing substrate 921 a pull tab 940 may be provided. The pull tab 940 may be integrally formed with the sealing substrate 921 or may be discrete and joined to the sealing substrate.

As shown, the cartridge comprises a major axis 971 and a minor axis 972. The pull tab 940, in order to facilitate packaging may have a length 945 generally parallel to the long axis 971 and a width 947 generally parallel to the minor axis 972. The length 945 and width 947 of the pull tab 940 may be any suitable value as described herein.

FIG. 10 shows an illustrated side cross-section of a cartridge 1001, having a reservoir 1004 containing volatile composition. The reservoir 1004 is enclosed by a membrane 1003a. The cartridge 1001 is not subjected to any compressive force, so the membrane 1003a is not deformed, and the reservoir has a fill level of volatile composition 1004a. In contrast, the cartridge 1002 is subjected to a compressive force F acting in a horizontal direction. The compressive force F deforms the membrane 1003b of the cartridge, thereby reducing a horizontal thickness of the reservoir 1004, causing an increase in the fill level of volatile composition 1004b.

Additional examples are shown below:

A. A volatile composition dispenser 1 comprising:
a housing 10 having a first end and an opposing second end, the housing comprising a front frame 100 and a rear frame 200 that are capable of moving away from or toward each other for opening and closing the housing, wherein the rear frame 200 comprises one or more rib elements 201; and
a cartridge (30, 930) configured for being positioned inside the housing 10, the cartridge comprising:
   a reservoir containing at least one liquid volatile composition; and
   a sealing substrate 33 for enclosing the reservoir,
wherein the at least one liquid volatile composition in the cartridge 30 is configured to be delivered to the atmosphere upon closing of the housing 10 when the front and rear frames move toward each other to bring the one or more rib elements 201 into contact with the cartridge 30 to cause rupture of the sealing substrate 33.

B. The volatile composition dispenser 1 as claimed in paragraph A, wherein the cartridge 30 is a refill cartridge, the refill cartridge being detachably (or releasably) disposed inside the housing such that it can be removed from within the housing 10 upon opening of the housing 10, and another refill cartridge can be placed inside the housing 10, followed by closing of the housing 10.

C. The volatile composition dispenser 1 as claimed in paragraph A or B, wherein the front frame 100 and the rear frame 200 are connected at the first end and are capable of pivotally moving away from or toward each other at the opposing second end for opening and closing the housing; wherein optionally the one or more rib elements 201 extend tangentially along the pivotal movement direction of the front and rear frames; wherein optionally the front frame 100 and the rear frame 200 are connected at the first end through a hinge structure, which comprises a knuckle element located on either of the front and rear frames and a pin element located on the other of the front and rear frames; wherein optionally the knuckle element or the pin element is molded as an integral part of the front or rear frame.

D. The volatile composition dispenser 1 as claimed in any one of paragraphs A to C, wherein the front frame 100 and the rear frame 200 further comprise a locking structure at the opposing second end of the housing for releasably interlocking the front and rear frames together upon closing of the housing; wherein optionally the locking structure is selected from the group consisting of latches, snap fits, clasps, catches, and magnets.

E. The volatile composition dispenser 1 as claimed in any one of paragraphs A to D, wherein each of the one or more rib elements 201 comprises a cartridge-contacting surface that is substantially free of protrusions; and wherein the cartridge further comprises one or more rupture elements 37 that are spatially aligned with the one or more rib elements 201, so that when the one or more rib elements 201 come closer or into contact with the cartridge 30, the one or more rib elements 201 press into the one or more rupture elements 37 and thereby causing rupture of the scaling substrate 33.

F. The volatile composition dispenser 1 as claimed in any one of paragraphs A to E, wherein the one or more rib elements 201 are located near the periphery of the rear frame 200; wherein optionally the one or more rib elements 201 comprise a first rib element closer to the first end of the housing 10 and a second rib element closer to the second opposing end of the housing 10.

G. The volatile composition dispenser 1 as claimed in any one of paragraphs A to F, wherein the housing 10 is a reusable housing that can be used multiple times with replaceable cartridges.

H. A method of delivering a volatile composition to an interior space, comprising the steps of:
(i) providing a volatile composition dispenser 1 as claimed in any one of paragraphs A to G in an interior space; and
(ii) activate the volatile composition dispenser 1 by moving the front and rear frames toward each other to close the housing 10.

I. A volatile composition dispenser 1 comprising:
a housing 10 having a first end and an opposing second end, the housing comprising a front frame 100 and a rear frame 200 that are capable of moving away from or toward each other for opening and closing the housing, the front frame 100 and/or rear frame 200 comprising one or more separation slots 720; and
a cartridge (30, 930) configured for being positioned inside the housing 10, the cartridge comprising:
   a reservoir containing at least one liquid volatile composition; and
   a sealing substrate 33 for enclosing the reservoir,
wherein the at least one liquid volatile composition in the cartridge 30 is configured to be delivered to the atmosphere via rupture and/or removal of sealing substrate 33.

J. The volatile composition dispenser 1 as provided in paragraph I, wherein the front frame 100 and the rear frame 200 are pivotable with respect to one another, wherein the separation slot 720 is disposed adjacent and upper portion 750 of the housing 10 and a pivot axis of the front frame 100 and the rear frame 200 is disposed in a lower portion 760 of the housing 10.

K. The volatile composition dispenser 1 as provided in paragraph I, wherein the front frame 100 and the rear frame 200 are pivotable with respect to one another, wherein the separation slot 720 is disposed adjacent a lower portion 760 and a pivot axis is disposed in an upper portion 750 of the housing 10.

L. The volatile composition dispenser 1 as provided in paragraph I, wherein the separation slot 720 is disposed on a side of the housing 10.

M. The volatile composition dispenser 1 as provided in any one of paragraphs I-L, wherein the separation slot is disposed at an interface 710 between the front frame 100 and the rear frame 200.

N. The volatile composition dispenser 1 as provided in any one of paragraphs I-M, wherein cartridge comprises a removable sealing substrate 33.

O. The volatile composition dispenser 1 as provided in paragraph N, wherein the sealing substrate 33 comprises a pull tab 945 disposed adjacent a seal area 935 on the cartridge 930.

P. The volatile composition dispenser 1 as provided in paragraph O, wherein the pull tab 945 comprises a length of from 20 mm or less, preferably about 15 mm or less, or more preferably about 10 mm or less.

Q. The volatile composition dispenser 1 as provided in any one of paragraphs O-P, wherein the pull tab 945 has a length of from about 2 mm to about 20 mm, preferably from about 5 mm to about 15 mm, or more preferably from about 5 mm to about 10 mm.

R. The volatile composition dispenser 1 as provided in any one of paragraphs O-Q, wherein the pull tab 945 has a width of about 20 mm or less, preferably about 15 mm or less or more preferably about 10 mm or less.

S. The volatile composition dispenser 1 as provided in any one of paragraphs O-R, wherein the pull tab 945 has a width of from about 2 mm to about 20 mm, preferably from about 5 mm to about 15 mm, or more preferably from about 5 mm to about 10 mm.

T. The volatile composition dispenser 1 as provided in any one of paragraphs O-S, wherein the front frame 100 and the rear frame 200 are pivotably connected, and wherein the separation slot is disposed on a side of the housing 10 and has a length which is at least 60 percent, more preferably at least 70 percent, or even more preferably at least 80 percent of a length 715 of an interface 710 between the front frame 100 and the rear frame 200.

U. The volatile composition dispenser 1 as provided in any one of paragraphs I and L-T, wherein the front frame 100 and the rear frame 200 are pivotably connected, and wherein the separation slot is disposed on a side of the housing 10 and has a length of from about 10 percent to about 60 percent, more preferably from about 10 percent to about 70 percent, or even more preferably from about 10 percent to about 80 percent of a length 715 of an interface 710 between the front frame 100 and the rear frame 200.

V. The volatile composition dispenser 1 as provided in any one of paragraphs O-S, wherein the front frame 100 and the rear frame 200 are pivotably connected, and wherein the separation slot is disposed opposite a pivot axis of the housing 10, and wherein the separation slot has a length which is has a length which is at least 60 percent, more preferably at least 70 percent, or even more preferably at least 80 percent of a length of a minor axis 972 of the cartridge 930.

W. The volatile composition dispenser 1 as provided in any one of paragraphs O-S and V, wherein the front frame 100 and the rear frame 200 are pivotably connected, and wherein the separation slot is disposed opposite a pivot axis of the housing 10, and wherein the separation slot has a length of from about 10 percent to about 60 percent, more preferably from about 10 percent to about 70 percent, or even more preferably from about 10 percent to about 80 percent of a length of a minor axis 972 of the cartridge 930.

X. The volatile composition dispenser 1 as provided in any one of paragraphs N-W, wherein the pull tab 945 extends through the separation slot.

Y. A method of delivering a volatile composition to an interior space, comprising the steps of:
(i) providing a volatile composition dispenser 1 as provided in any one of paragraphs I to W in an interior space; and
(ii) activating the volatile composition dispenser 1 by rupturing or removing the sealing substrate, wherein removal of the sealing substrate can be prior to insertion of the cartridge into the housing or pulling the sealing substrate through the separation slot.

Z. The method of delivering a volatile composition to an interior space according to paragraph Y, wherein the rear frame 200 comprises one or more rib elements 201 which rupture the sealing substrate 33 upon closure of the housing 10.

AA. A volatile composition dispenser 1 comprising:
a housing 10 having a first end and an opposing second end, the housing comprising a front frame 100 and a rear frame 200 that are capable of moving away from or toward each other for opening and closing the housing, the front frame 100 and/or rear frame 200 comprising one or more separation tabs 810/820; and
a cartridge (30, 930) configured for being positioned inside the housing 10, the cartridge comprising:
   a reservoir containing at least one liquid volatile composition; and
   a sealing substrate 33 for enclosing the reservoir,
wherein the at least one liquid volatile composition in the cartridge 30 is configured to be delivered to the atmosphere via rupture and/or removal of sealing substrate 33.

AB. The volatile composition dispenser 1 as provided in paragraph AA, wherein the front frame 100 and the rear frame 200 are pivotable with respect to one another, wherein the separation tabs 810 and 820 are disposed adjacent and upper portion 750 of the housing 10 and a pivot axis of the front frame 100 and the rear frame 200 is disposed in a lower portion 760 of the housing 10.

AC. The volatile composition dispenser 1 as provided in paragraph AA, wherein the front frame 100 and the rear frame 200 are pivotable with respect to one another, wherein the separation tabs 810 and 820 are disposed adjacent a lower portion 760 and a pivot axis is disposed in an upper portion 750 of the housing 10.

AD. The volatile composition dispenser 1 as provided in any one of paragraphs AA to AC, wherein the front frame 100 comprises separation tab 810 and the rear frame 200 comprises separation slot 820.

AE. The volatile composition dispenser 1 as provided in any one of paragraphs AA-AD, wherein cartridge comprises a removable sealing substrate 33.

AF. The volatile composition dispenser 1 as provided in paragraph AE, wherein the sealing substrate 33 comprises a pull tab 945 disposed adjacent a seal area 935 on the cartridge 930.

AG. The volatile composition dispenser 1 as provided in paragraph AF, wherein the pull tab 945 comprises a length of from 20 mm or less, preferably about 15 mm or less, or more preferably about 10 mm or less.

AH. The volatile composition dispenser 1 as provided in any one of paragraphs AF-AG, wherein the pull tab 945 has a length of from about 2 mm to about 20 mm, preferably from about 5 mm to about 15 mm, or more preferably from about 5 mm to about 10 mm.

AI. The volatile composition dispenser 1 as provided in any one of paragraphs AF-AH, wherein the pull tab 945 has a width of about 20 mm or less, preferably about 15 mm or less or more preferably about 10 mm or less.

AJ. The volatile composition dispenser 1 as provided in any one of paragraphs AF-AI, wherein the pull tab 945 has a width of from about 2 mm to about 20 mm, preferably from about 5 mm to about 15 mm, or more preferably from about 5 mm to about 10 mm.

AK. A method of delivering a volatile composition to an interior space, comprising the steps of:
(i) providing a volatile composition dispenser 1 as provided in any one of paragraphs AA to AJ in an interior space; and
(ii) activating the volatile composition dispenser 1 by rupturing or removing the sealing substrate, wherein removal of the sealing substrate can be prior to insertion of the cartridge into the housing slot.

AL. The method of delivering a volatile composition to an interior space according to paragraph AK, wherein the rear frame 200 comprises one or more rib elements 201 which rupture the sealing substrate 33 upon closure of the housing 10.

AM. A cartridge (30, 930) for a volatile composition dispenser 1, the cartridge comprising: a container 32 having a reservoir 321, a sealing substrate 33 which sealably covers the reservoir 321 and a membrane, wherein sealing substrate 33 is rupturable or removable.

AN. The cartridge (30, 930) for a volatile composition dispenser 1, according to paragraph AM, wherein the substrate is rupturable.

AO. The cartridge (30, 930) for a volatile composition dispenser 1, according to paragraph AN, wherein substrate comprises a film, foil, or laminate material.

AP. The cartridge (30, 930) for a volatile composition dispenser 1, according to paragraph AM, wherein the sealing substrate is removable.

AQ. The cartridge (30, 930) for a volatile composition dispenser 1, according to paragraph AP, wherein the sealing substrate 33 comprises a pull tab 945 disposed adjacent a seal area 935 on the cartridge 930.

AR. The cartridge (30, 930) for a volatile composition dispenser 1, according to any one of paragraphs AP to AQ, wherein the pull tab 945 comprises a length of from 20 mm or less, preferably about 15 mm or less, or more preferably about 10 mm or less.

AS. The cartridge (30, 930) for a volatile composition dispenser 1, according to any one of paragraphs AP to AR, wherein the pull tab 945 has a length of from about 2 mm to about 20 mm, preferably from about 5 mm to about 15 mm, or more preferably from about 5 mm to about 10 mm.

AT. The cartridge (30, 930) for a volatile composition dispenser 1, according to any one of paragraphs AP to AS, wherein the pull tab 945 has a width of about 20 mm or less, preferably about 15 mm or less or more preferably about 10 mm or less.

AU. The cartridge (30, 930) for a volatile composition dispenser 1, as provided in any one of paragraphs AP to AT, wherein the pull tab 945 has a width of from about 2 mm to about 20 mm, preferably from about 5 mm to about 15 mm, or more preferably from about 5 mm to about 10 mm.

AV. A method of delivering a volatile composition to an interior space, comprising the steps of:
(i) providing a cartridge for a volatile composition dispenser 1 as provided in any one of paragraphs AP to AU; and
(ii) removing the sealing substrate and inserting the cartridge into the front frame 100 of the housing 10.

AW. The method of delivering a volatile composition to an interior space, as provided in paragraph AV, wherein removal of the sealing substrate occurs prior to insertion of the cartridge into a front frame 100 of a housing 10.

AX. The method of delivering a volatile composition to an interior space as provide in paragraph AV, wherein removal of the sealing substrate occurs after insertion of the cartridge into a front frame 100 of the housing 10.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular configurations of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A volatile composition dispenser comprising:
a housing having a first end and an opposing second end, the housing comprising a front frame and a rear frame that are capable of moving away from or toward each other for opening and closing the housing, wherein the rear frame comprises one or more rib elements; and
a cartridge configured to be positioned inside the housing, the cartridge comprising:
a reservoir containing at least one liquid volatile composition;
a sealing substrate for enclosing the reservoir,
wherein:
rupture or removal of the sealing substrate enables evaporation of the at least one liquid volatile composition from the cartridge; and
the rib elements are configured to apply a compressive force on the cartridge when the housing is in a closed state and the cartridge is positioned within the housing.

2. The volatile composition dispenser according to claim 1, wherein the cartridge comprises a membrane for enclosing the reservoir, and the rib elements are configured to apply the compressive force on the membrane, thereby causing a deformation of the membrane.

3. The volatile composition dispenser according to claim 1, wherein the rib elements are configured to apply the compressive force on the cartridge for substantially an entire lifetime of a cartridge positioned within the housing.

4. The volatile composition dispenser according to claim 1, wherein:
the housing comprises a locking structure for releasably locking the housing in a closed state; and
when a cartridge is positioned within the housing and the housing is in a closed state, the cartridge applies an opening force on the housing.

5. The volatile composition dispenser according to claim 4, wherein when the locking structure is released the opening force causes the front frame and rear frame to move away from each other.

6. The volatile composition dispenser according to claim 5, wherein:
the front and rear frames are pivotally connected at a first end and capable of moving away from or toward each other at an opposing second end; and
the locking structure is located at the second end.

7. The volatile composition dispenser according to claim 1, wherein the compressive force causes a reduction in a thickness of the reservoir, as compared to a thickness of the reservoir before being subjected to the compressive force.

8. The volatile composition dispenser according to claim 1, wherein:
the cartridge comprises a membrane for enclosing each of the sealing substrate and the reservoir;
the sealing substrate is configured to be ruptured by the compressive force, without the cartridge being touched by a user,
wherein the compressive force, when applied, actuates a rupture mechanism that is configured to rupture the sealing substrate,
wherein the cartridge comprises one or more rupture elements that are spatially aligned with the one or more rib elements, so that when the one or more rib elements come into contact with the cartridge, the one or more rib elements applies the compressive force on the one or more rupture elements and thereby cause rupture of the sealing substrate.

9. The volatile composition dispenser according to claim 1, wherein the cartridge is configured to be held within the housing without adhesion or attachment between the cartridge and housing.

10. The volatile composition dispenser according to claim 1, wherein the cartridge is configured to be removed from the housing without being touched by a user.

11. The volatile composition dispenser according to claim 1, wherein the housing and cartridge are configured such that when a cartridge located within the housing has reached end of life, the cartridge may be removed from the housing by the steps:
(i) opening the housing; and
(ii) upending the housing,
without a step of disengaging or disadhering the cartridge from the housing.

12. The volatile composition dispenser according to claim 1, wherein the front frame comprises a window portion configured to receive the reservoir of the cartridge.

13. The volatile composition dispenser according to claim 1, wherein the cartridge is a replaceable refill cartridge.

14. The volatile composition dispenser according to claim 1, wherein the front frame and the rear frame are connected at the first end and are capable of pivotally moving away from or toward each other at the opposing second end for opening and closing the housing;
wherein the one or more rib elements extend tangentially along the pivotal movement direction of the front and rear frames; wherein the front frame and the rear frame are connected at the first end through a hinge structure, which comprises a knuckle element located on either of the front and rear frames and a pin element located on the other of the front and rear frames; wherein the knuckle element or the pin element is molded as an integral part of the front or rear frame.

15. The volatile composition dispenser according to claim 1, wherein the front frame and the rear frame further comprise a locking structure at the opposing second end of the housing for releasably interlocking the front and rear frames together upon closing of the housing; wherein the locking structure is selected from the group consisting of latches, snap fits, clasps, and catches.

16. The volatile composition dispenser according to claim 1, wherein each of the one or more rib elements comprises a cartridge-contacting surface that is substantially flat or rounded.

17. The volatile composition dispenser according to claim 16, wherein the cartridge further comprises one or more rupture elements that are spatially aligned with the one or more rib elements, so that when the one or more rib elements come closer or into contact with the cartridge, the one or more rib elements apply the compressive force on the one or more rupture elements and thereby causing rupture of the sealing substrate.

18. The volatile composition dispenser according to claim 1, wherein the one or more rib elements are located near a periphery of the rear frame; wherein the one or more rib elements comprise a first rib element closer to the first end of the housing and a second rib element closer to the second opposing end of the housing.

19. The volatile composition dispenser according to claim 1, wherein the housing is a reusable housing that can be used multiple times with replaceable cartridges.

20. The volatile composition dispenser according to claim 1, wherein the cartridge is not able to rotate within the housing.
